(19) United States
(12) Patent Application Publication
(10) Pub. No.: US 2002/0107291 A1
(43) Pub. Date: Aug. 8, 2002

(54) Clear, injectable formulation of an anesthetic compound

(76) Inventor: Vincenzo De Tommaso, Basiglio (IT)

Correspondence Address: DARBY & DARBY, 805 THIRD AVENUE, 27TH FLR., NEW YORK, NY, 10022, US

(21) Appl. No.: 09/976,450

(22) Filed: Oct. 11, 2001

Related U.S. Application Data
(63) Continuation-in-part of application No. 09/424,691, filed on Jul. 18, 2000.

(30) Foreign Application Priority Data
May 14, 1998 (EP) PCT/EP98/03004
May 26, 1997 (CH) 1224/97

(51) Int. Cl. A61K 31/05

(52) U.S. Cl. 514/731

(57) ABSTRACT

The present invention is directed to a clear, injectable pharmaceutical composition including propofol, a pharmaceutically acceptable salt of a bile acid and a lecithin. The present invention is further related to a process for the preparation of a clear, injectable pharmaceutical composition including propofol, a pharmaceutically acceptable salt of a bile acid and a lecithin.

The present invention relates to a clear, injectable, pharmaceutical formulation of propofol.

### BACKGROUND OF THE INVENTION

Propofol, whose chemical name is 2,6-bis-(1-methylethyl)phenol, is a known anaesthetic, largely used for general anesthesia.

The propofol formulation which is present on the market is a non-trasparent, white, oil-in-water emulsion. Similar formulations are described for example in U.S. Pat. No. 4,798,846 and in GB 2,298,789.

Other injectable propofol preparations have been described. More particularly, WO 96/32135 discloses a pharmaceutical composition in which propofol is used as an inclusion complex with 2-hydroxypropyl-p-cyclodextrine while WO 97/10814 discloses the use of nanodispersions of propofol to be administered by intravenous route.

### SUMMARY OF THE INVENTION

The present invention is directed to an aqueous, injectable pharmaceutical composition including propofol, a pharmaceutically acceptable salt of a bile acid and a lecithin.

Another embodiment of the present invention is a process for the preparation of the aqueous, injectable pharmaceutical composition including propofol, a pharmaceutically acceptable salt of a bile acid and a lecithin. The process includes the following steps: (a) adding lecithin to an aqueous solution of the pharmaceutically acceptable salt of the bile acid, the solution having a pH from 4.5 to 6.5; (b) heating the aqueous dispersion at a temperature from 35° to 85° C.; (c) adding propofol, previously heated at a temperature from 35° to 85° C., to the solution obtained in step (b); and (d) cooling and adding water to the final volume.

It has now been found that a transparent injectable formulation of propofol may be obtained by mixing propofol with a bile acid and with a lecithin. More particularly, said formulation presents noteworthy advantages in respect of the presently marketed formulation. Such a formulation is clear, and, hence, the presence of foreign particles, such as glass residues, fibers, undissolved substances and the like, inside the vials or bottles can be easily controlled. This feature is very important for product safety because, in general, the ready-for-use injectable solutions and, with greater reason, those exclusively used intravenous administration, as is the case of propofol, must not contain any foreign particles.

Furthermore, the present injectable formulation may be diluted in most of the solutions for infusion, thus allowing the anesthetist physician to dose the drug with better precision and to administer it with a greater regularity in order to obtain a more precise and safer effect.

Another very important advantage is the fact that the present formulation is stable within a temperature range from +2° to +35° C., which is broader than the stability temperature range (+2 to +25° C.) of the presently marketed formulation.

Moreover, the production of the formulation of the present invention does not require any particular or sophisticated apparatus, but it is sufficient to use normal equipment for the production of pharmaceutical formulations for injectable use.

Thus, it is another object of the present invention to provide an aqueous, injectable pharmaceutical composition comprising:

(a) propofol;

(b) a pharmaceutically acceptable salt of a bile acid;

(c) a lecithin.

### DETAILED DESCRIPTION OF THE INVENTION

The bile salt, incorporated in the injectable formulation of the invention, is advantageously selected from the group consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid, the preferred one being glycocholic acid.

The pharmaceutically acceptable salts of the bile acids may be advantageously selected from the group consisting of the sodium, potassium, calcium, magnesium or ammonium salts. The sodium salt being preferred. Sodium glycocholate is the particularly preferred pharmaceutically acceptable salt of a bile acid.

A lecithin may be soybean lecithin or egg lecithin.

The use of a salt of a cholanic acid in admixture with lecithin for the preparation of mycellar solutions of non-steroidal anti-inflammatory compounds, in order to reduce or suppress the local irritations and the haemolytic effects deriving from the parenteral administration of aqueous solutions of the drugs, is described in EP-A-280887.

In the aqueous pharmaceutical formulation of the present invention, propofol is present in an amount from 5 mg to 50 mg per 1 ml of formulation, advantageously from 5 mg to 25 mg per ml of formulation, more preferably from 10 mg to 20 mg per ml of formulation.

The pharmaceutically acceptable salt of the bile acid is present in the aqueous pharmaceutical formulation in an amount, referred to the free acid, from 30 to 50 mg per 1 ml of formulation, preferably from 35 to 45 mg per ml of formulation. Lecithin is present in an amount from 30 to 50 mg, preferably from 30 to 45 mg per ml of formulation, much preferably from 35 to 40 mg per ml of formulation. Soybean lecithin is the preferred lecithin.

According to an advantageous embodiment of the present invention, the aqueous, injectable pharmaceutical composition comprises about 10 mg of propofol, 38.2 mg of glycocholic acid and 37.8 mg of soybean lecithin per ml of formulation; according to a further advantageous embodiment of the present invention, the aqueous, injectable pharmaceutical composition comprises about 20 mg of propofol, 43.7 mg of glycocholic acid mg and 37.8 mg of soybean lecithin per ml of formulation.

According to a particularly advantageous embodiment of the present invention, in this aqueous, injectable pharmaceutical composition, said bile acid salt is sodium glycocholate and said lecithin is soybean lecithin.

The water used in the present formulation is water for injectable preparations. For the manufacture of the present pharmaceutical formulation, the bile acid salt may be straightforwardly used as starting material or the free acid may be previously converted to an appropriate salt with a suitable alkalinizing agent which may be, for example, an alkali metal hydroxide such as sodium, potassium or lithium hydroxide, an alkaline-earth metal hydroxide, such as calcium or magnesium hydroxide, a metal oxide such as magnesium or aluminum oxide, a carbonic acid salt, such as sodium or potassium carbonate, sodium or potassium bicarbonate, a phosphoric acid salt, such as sodium, potassium or calcium phosphate, for example trisodium phosphate.

It is another object of the present invention to provide a process for the preparation of an aqueous, injectable pharmaceutical composition as mentioned above, which comprises:

(a) adding lecithin to an aqueous solution of the pharmaceutically acceptable salt of the bile acid, said solution having a pH from 4.5 to 6.5, preferebly from 5.8-6.3;

(b) heating the aqueous dispersion to a temperature from 35° to 85° C. for 60 minutes;

(c) adding propofol, previously heated at a temperature from 35° to 85° C., to the solution obtained in step (b), heated at a temperature from 35° to 85° C.;

(d) cooling and adding water to reach the final volume. More particularly, the present invention concerns a process for the preparation of an aqueous, injectable pharmaceutical composition containing propofol, a pharmaceutically acceptable salt of a bile acid and a lecithin, as illustrated above, which comprises:

(a) adding lecithin to an aqueous solution of the pharmaceutically acceptable bile acid salt, said solution having a pH from 4.5 to 6.5;

(b) heating the aqueous dispersion at a temperature from 35° to 85° C. until solution is complete;

(c) adding propofol, previously heated to the temperature from 35° to 85° C., to said solution;

(d) cooling to room temperature and adding water until the final volume is reached, all the steps being carried out in substantial absence of oxygen. The expression “substantial absence of oxygen” means that the solution, during the process, should have a content of oxygen not higher than 1 part per million (p.p.m.), preferably not higher than 0.5 p.p.m.

As set forth above in step (a), the pharmaceutically acceptable salt of the bile acid, preferably sodium glycocolate, may be dissolved in water as such or prepared in situ by salification of the bile acid, preferably glycocholic acid, with the selected base, preferably sodium hydroxide. In this latter case, the bile acid, preferably glycocholic acid, is added to an aqueous solution of the base, preferably sodium hydroxide, by adjusting the pH of the solution thus obtained with a pharmaceutically acceptable acid, preferably hydrochloric acid, in order to render the pH compatible with an intravenous administration. The pH is kept at a value from 4.5 to 6.5, advantageously from 5 to 6, preferably about 5.5.

Step (a) is normally carried out at room temperature (20° to 25° C.), but a higher temperature, for example of about 30° C. is also acceptable. The medium is advantageously kept under substantial absence of oxygen by using any technique for removing it, for example by bubbling an inert gas, preferably nitrogen, in the medium and by keeping the medium under inert atmosphere throughout the process. The content of oxygen may be measured according to known methods (for example using an oxygen-sensitive electrode) and kept not higher than 1 p.p.m., preferably lower than 0.5 p.p.m. Lecithin, preferably soybean lecithin, is added under strong stirring, advantageously in an inert atmosphere, preferably under nitrogen stream.

In step (b), the mixture is heated at a temperature of from 35° to 85° C. in order to obtain complete dissolution.

Usually, a temperature from 35° to 60° C., preferably from 450 to 50° C., is used. Since bile acids and their salts are often surfactants, a foam may be obtained, which dissolves if the mixture is let to stand at rest, advantageously always in substantial absence of oxygen.

In step (c), to the solution thus obtained, heated to 35° to 85° C., preferably at 55° to 60° C., propofol, previously heated at the same temperature, is added under stirring and advantageously in an inert atmosphere, preferably under nitrogen stream.

In step (d) the clear solution thus obtained, if necessary homogeneized, is cooled to room temperature (22 to 25° C.) and diluted with water until the desired volume is reached, preferably by keeping the oxygen concentration of the medium very low, advantageously not higher than 1 p.p.m., preferably lower than 0.5 p.p.m.

The solution thus obtained, when submitted to the conventional operations of pharmaceutical technique for the manufacture of injectable preparations, preferably kept in hermetically closed vessels, is ready for medical use. Preferably, the formulation according to the present invention contains oxygen at a concentration not higher than 0.5 p.p.m.. In the vessel containing it (vial or bottle) the head space contains oxygen in an amount preferably not higher than 1%.

### EXAMPLE 1

In a stainless steel reactor equipped with an heating shell, 186 mls of water for injectable preparation were introduced and nitrogen was bubbled thereinto to a concentration of dissolved oxygen lower than 0.5 p.p.m.. Keeping the water temperature at about 25° C., 4.8 grams of sodium hydroxide were added to the water, under gentle stirring and nitrogen stream. At complete dissolution, a control of the oxygen concentration, to be kept lower than 0.5 p.p.m., was made, then 54.6 grams of glycocholic acid were quickly added in one portion by keeping the mixture at about 30° C. under strong stirring and nitrogen atmosphere. After dissolution, the pH was in the range of 10 to 12. After solubilization of the glycocholic acid, the pH was adjusted to 5.45 to 5.5 with IN hydrochloric acid, by adding said acid slowly and keeping the solution under strong stirring and nitrogen atmosphere (oxygen concentration lower than 0.5 p.p.m.) To the solution of sodium glycocholate, 75.6 grams of soybean lecithin were slowly added. Keeping the solution under strong stirring and nitrogen stream, the suspension was then heated at a temperature of 45 to 50° C. under moderate stirring and nitrogen atmosphere until the formation of a great amount of foam was observed. The solution was cooled to room temperature and stood for 18 hours under nitrogen pressure, wereafter the complete solubilization of the soybean lecithin was controlled. The solution was heated at 55 to 60° C. and submitted for 10 minutes to the action of a homogeneizer, under strong nitrogen stream. An amount of 10 g of propofol, previously heated to 60° C., was slowly poured into the previously obtained solution, by keeping it under homogeneization and nitrogen stream, at a temperature of 65 to 70° C. The solution was homogeneized until a sample of solution, diluted 1:1 v/v with water for injectable preparations, showed clear by the naked eye. The solution was cooled to 25° C. by adding 610 ml of water for injectable preparations, very slowly, into the solution at a temperature of 25° C. with a content of dissolved oxygen lower than 0.5 p.p.m. The solution was then kept under gentle stirring and nitrogen stream until the content of dissolved oxygen resulted lower than 0.5 p.p.m. Then, vacuum was made in the reactor in order to eliminate the gas dissolved in the solution and water for injectable preparation, with a content of dissolved oxygen lower than 0.5 p.p.m., was added to a volume of 1000 ml. The solution was kept under gentle stirring and nitrogen stream; the value of the pH was controlled in order to kept it at 6.0 to 6.3 (if necessary, the value must be adjusted with 0.2% hydrochloric acid or with 0.2% sodium hydroxide). The amount of dissolved oxygen was controlled and the nitrogen bubbling was continued until an oxygen concentration lower than 0.5 p.p.m. was obtained. The solution was filtered in a sterile unit (class 100) through a 0.22-micron porous membrane, type Durapore® by Millipore, previously controlled and approved for its integrity. Vials or bottles were filled under nitrogen atmosphere, by controlling the amount of residual oxygen in the head space of the vial or bottle in order to keep it lower than 1%.

### STABILITY ASSAYS

After the initial measurement (time 0=T₀) controls were made after 30 days (T₃₀) and after 60 days (T₆₀) from T₀.

For the detection of propofol, a method by HPLC, performed with inverse phase column and a UV detector was set up. The specificity and response linearity study in the concentration range of the vials gave satisfactory results to perform the stability control suitably.

### EXPERIMENTAL

### MATERIALS AND METHODS

Reagents

Acetonitrile for HPLC, Merck—Darmstadt

Deionized water from equipment “Maxima ultra pure water”, Elga

Propofol Standard, Archimica—code 61005, batch No. 95005-0-01 (purity grade: 99.8%; density: 0.955 g/ml).

Standard Solutions

Standard solution of propofol: in a 100-ml volumetric flask, about 20.0 mg, exactly weighed, of pure product are mixed with water to volume.

Soluzions of propofol samples in vials: the content of two vials are poured in a perfectly dried flask. 2 ml of the liquid of the vials are pipetted, by a glass pipette, poured in a 100-ml volumetric flask and brought to volume with water.

Equipment

HPLC CM 4000, Milton Roy, equipped with valve Rheodyne 7125 with a 10-μl loop;

detector U. V. Spectromonitor 3100, Milton Roy, with variable wavelength;

integrator Mega 2, Carlo Erba, with paper speed=0.5 cm/min.;

column: Lichrospher 100 RP-18 (125 cm×4 mm i. d.—5 μ particle size), Merck—Darmstadt;

precolumn: RP 18, Merck—Darmstadt.

Chromatographic Conditions

Mobile phase: acetonitrile/water=60:40 v/v

Flow: 1 ml/min.

Detector wavelength: 270 nm.

Average elution time of propofol: 4.30±1.00.

Analytical detection

For the detection of propofol concentration in the vials, the standard solution of propofol is analyzed by repeating the analysis four times. The solutions of propofol samples in vials are analyzed immediately after the detection of the standard, by repeating the analysis twice. From the comparison of the average areas drawn for the peaks of the propofol, the concentration in mg/ml of the active principle in the solution of the vials is calculated.

The results summarized in Tables 1, 2 and 3 show that the solution object of the present invention remains stable and clear for a period of at least 150 days from the preparation date, within a broad temperature range.

TABLE 1
PROPOFOL 10 mg/ml - Temperature: 5° C.

| ANALYSIS | | CONCENTRATION | | |
|---|---|---|---|---|
| DATE | DAYS | mg/ml | pH | DEGRADATION |
| 16.10.96 | 0 | 8.57 | 6.00 | 0 |
| 19.11.96 | 30 | 8.58 | 6.17 | +0.12 |
| 17.12.96 | 60 | 8.62 | 6.12 | +0.58 |
| 10.02.97 | 120 | 8.48 | n. d. | −1.05 |
| 27.03.97 | 150 | 8.25 | 6.15 | −3.70 |

TABLE 2
PROPOFOL 10 mg/ml - Temperature: 25° C.

| ANALYSIS | | CONCENTRATION | | |
|---|---|---|---|---|
| DATE | DAYS | mg/ml | pH | DEGRADATION |
| 16.10.96 | 0 | 8.57 | 6.00 | 0 |
| 19.11.96 | 30 | 8.46 | 6.16 | −1.28 |
| 17.12.96 | 60 | 8.38 | 6.13 | −2.21 |
| 10.02.97 | 120 | n. d. | n. d. | n. d. |
| 27.03.97 | 150 | n. d. | n. d. | n. d. |

TABLE 3
PROPOFOL 10 mg/ml - Temperature: 40° C.

| ANALYSIS | | CONCENTRATION | | |
|---|---|---|---|---|
| DATE | DAYS | mg/ml | pH | DEGRADATION |
| 16.10.96 | 0 | 8.57 | 6.00 | 0 |
| 19.11.96 | 30 | 8.52 | 6.19 | −0.58 |
| 17.12.96 | 60 | 8.38 | 6.15 | −2.21 |
| 10.02.97 | 120 | 8.34 | 6.21 | −2.68 |
| 27.03.97 | 150 | 8.26 | 6.19 | −3.60 |

n. d. = not determined

### EXAMPLE 2

The process disclosed in example 1 was repeated by using the amounts of propofol, glycocholic acid, soybean lecithin, water, sodium hydroxide and hydrochloric acid reported here-below:

| | | |
|---|---|---|
| Propofol | 10.0 | mg; |
| Glycocholic acid | 38.2 | mg; |
| Soybean lecithin | 37.8 | mg; |
| Water for injections up to | 1.0 | ml; |
| Sodium Hydroxide | 3.2 | mg; |

| | |
|---|---|
| Hydrochloric acid up to | pH 5.8-6.3; |

also in this case a clear aqueous injectable formulation was obtained.

### EXAMPLE 3

The process disclosed in example 1 was repeated by using the amounts of propofol, glycocholic acid, soybean lecithin, water, sodium hydroxide and hydrochloric acid reported here-below:

| | | |
|---|---|---|
| Propofol | 20.0 | mg; |
| Glycocholic acid | 43.7 | mg; |
| Soybean lecithin | 37.8 | mg; |
| Water for injections up to | 1.0 | ml; |
| Sodium Hydroxide | 3.8 | mg; |

| | |
|---|---|
| Hydrochloric acid up to | pH 5.8-6.3; |

also in this case a clear aqueous injectable formulation was obtained.

1. A process for the preparation of a clear, aqueous injectable pharmaceutical formulation of propofol, comprising the following steps:
(a) adding a lecithin to an aqueous solution of a pharmaceutically acceptable bile acid salt, the aqueous solution having a pH from 4.5 to 6.5;
(b) heating the aqueous dispersion obtained in step (a) to a temperature from 35° to 85° C. to dissolve the lecithin;
(c) dissolving any foam which is obtained in step (b);
(d) heating the dispersion obtained in step (c) to a temperature from 35° to 85° C;
(e) adding propofol to the dispersion obtained in step (d);
(f) cooling the dispersion obtained in step (e) and adding water to obtain the clear, aqueous formulation in the desired final volume;
wherein said formulation contains from 5 to 50 mg of propofol/ml, from 30 to 45 mg of lecithin/ml and from 30 to 50 mg of the pharmaceutically acceptable bile acid salt/ml.

2. The process according to claim 1, wherein said formulation contains from 5 to 25 mg of propofol/ml, from 35 to 40 mg of lecithin/ml and from 35 to 45 mg of the pharmaceutically acceptable bile acid salt/ml.

3. The process according to claim 1, wherein said formulation contains from 10 to 20 mg of propofol/ml.

4. The process according to claim 1, carried out in substantial absence of oxygen.

5. The process according to claim 1, wherein the bile acid salt is sodium glycocholate.

6. The process according to claim 1, wherein the lecithin is soybean lecithin.

7. The process according to claim 3, wherein the substantial absence of oxygen is obtained by bubbling an inert gas into the solution in step (a) and maintaining the aqueous media in steps (a)-(d) under the inert atmosphere.

8. The process according to claim 6, wherein the inert gas is nitrogen.

9. The process according to claim 1, wherein the aqueous media in steps (a)-(d) have oxygen content no higher than 1 p.p.m.

10. The process according to claim 1, wherein the aqueous solution in step (a) has a pH from 5.8 to 6.3.

11. A clear, aqueous injectable pharmaceutical formulation of propofol prepared by the process set forth in claim 1.

12. A clear, aqueous injectable pharmaceutical formulation of propofol prepared by the process set forth in claim 2.

13. A clear, aqueous injectable pharmaceutical formulation of propofol prepared by the process set forth in claim 3.

14. A clear, aqueous injectable pharmaceutical formulation of propofol prepared by the process set forth in claim 4.

15. A clear, aqueous injectable pharmaceutical formulation of propofol prepared by the process set forth in claim 5.

16. A clear, aqueous injectable pharmaceutical formulation of propofol prepared by the process set forth in claim 6.

17. A clear, aqueous injectable pharmaceutical formulation of propofol prepared by the process set forth in claim 7.

18. A clear, aqueous injectable pharmaceutical formulation of propofol prepared by the process set forth in claim 8.

19. A clear, aqueous injectable pharmaceutical formulation of propofol prepared by the process set forth in claim 9.

20. A clear, aqueous injectable pharmaceutical formulation of propofol prepared by the process set forth in claim 10.
